(19) United States
(12) Patent Application Publication
(10) Pub. No.: US 2015/0182959 A1
(43) Pub. Date: Jul. 2, 2015

(54) RELATING TO EPOXIDATION CATALYSTS

(71) Applicant: SHELL OIL COMPANY, Houston, TX (US)

(72) Inventor: Nicoleta Cristina NENU, Villanova, PA (US)

(21) Appl. No.: 14/580,493

(22) Filed: Dec. 23, 2014

(30) Foreign Application Priority Data
Dec. 30, 2013 (EP) 13199854.4

(51) Int. Cl. B01J 37/02; B01J 21/06; C07C 1/24; B01J 37/08; C07D 301/19; C07C 29/48

(52) U.S. Cl. CPC B01J 37/0201; C07D 301/19; C07C 29/48; C07C 1/24; B01J 37/08; B01J 37/0203; B01J 21/063

(57) ABSTRACT

A process for the preparation of a titanium-based catalyst active in epoxidation reactions, which process comprises the steps of:
(a) impregnating a silica carrier with a liquid solution of a titanium compound in an inorganic solvent system, to form an impregnated silica carrier bearing the solution of the titanium compound;
(b) drying the impregnated silica carrier obtained in step (a);
(c) calcining the product obtained in step (b) at a temperature of at most 750° C.; and
(d) silylating the product obtained in step (c), to give a titanium-based catalyst active in epoxidation reactions.

This non-provisional application claims the benefit of European Application No. 13199854.4 filed Dec. 30, 2013 which is hereby incorporated by reference in its entirety.

### FIELD OF THE INVENTION

This invention relates to a process for the preparation of a titanium-based catalyst active in epoxidation reactions, a titanium-based catalyst obtainable by this process, and a process for the preparation of an epoxide which uses the titanium-based catalyst.

### BACKGROUND OF THE INVENTION

Titanium-based catalysts are known to be useful in the preparation of epoxides from alkenes using a hydroperoxide. Processes for the preparation of titanium-based catalysts which are active in epoxidation reactions are also known; titanium sites are commonly created on silica surfaces by gas phase impregnation or by impregnation using organic solvents.

EP 0345856 A discloses a process for making a titanium catalyst which is suitable for epoxidising alkenes using a hydroperoxide, wherein a solid silica and/or inorganic silicate is impregnated with gaseous titanium tetrachloride. WO2004050233 describes a process for the preparation of an epoxidation catalyst, which process comprises impregnating a silicon containing carrier with a gas stream consisting of titanium halide.

Other existing methods for preparing epoxidation catalysts involve dissolving a titanium species in an organic solvent for emplacement on a carrier. Such a process is described in e.g. U.S. Pat. No. 6,187,934, which describes using alcohols, ketones, ethers or esters as solvent; and U.S. Pat. No. 6,011,162, which describes using non-oxygenated hydrocarbon solvents such as heptane.

Both gas phase impregnation and impregnation using organic solvents have disadvantages.

Both of these methods are difficult to conduct on a large scale. Furthermore, titanium emplacement via gas phase impregnation requires very expensive assets (due to inherent corrosion problems), while titanium emplacement via organic impregnation involves burn off of excess solvent which can cause temperature runaways and releases large amounts of CO₂. These issues make the titanium emplacement step costly and environmentally unfriendly.

U.S. Pat. No. 3,829,392 A discloses that the efficiency of olefin epoxidation catalysts comprising an inorganic oxygen compound of silicon in chemical combination with certain metal oxides or hydroxides is improved when such catalysts are treated prior to use by contact with an organic silylating agent at elevated temperatures.

One catalyst preparation method described in U.S. Pat. No. 3,829,392 at column 2, line 70 to column 3, line 4 is said to involve impregnation of a siliceous support with a suitable metal-containing solution followed by heating, cogelling the metal hydroxide and silica, and by calcining together a mixture of inorganic siliceous solid and metal oxides at elevated temperatures. However, U.S. Pat. No. 3,829,392 A indicates that the method of catalyst preparation is not critical to the functioning or effectiveness of the invention described therein.

Example III in U.S. Pat. No. 3,829,392 describes the preparation of a silylated catalyst from silica gel and an aqueous solution of titanium tetrachloride and oxalic acid. After impregnation, the silica gel was dried and then calcined at 800° C. to produce a titania/silica product and said product was subjected to subsequent silylation.

EP1005907 A1 describes a catalyst for the epoxidation of unsaturated hydrocarbons, wherein said catalyst comprises finely divided gold particles immobilised on a titanium-containing oxide and is subjected to silylation or a hydrophobizing treatment.

Example 2 in EP1005907 A1 discloses the immersion of silica in an aqueous solution of titanium bisammonium lactate dihydroxide. After distillation of excess water, the residue was dried and then calcined at 900° C. to give a titanium/silica composite oxide (a titania-silica product). Said titania-silica product was then treated with an aqueous solution of chloroauric acid, in order to produce, after work-up, a catalyst comprising a titanium-containing oxide having ultra-fine gold particles supported thereon.

It will be appreciated that as the products produced in the afore-mentioned examples of U.S. Pat. No. 3,829,329 and EP1005907 A1 are titania-silica products, the titanium therein will exist in a fully octahedral geometry (coordination number 6).

It is an aim of the invention to provide an alternative process for the preparation of a titanium-based catalyst exhibits good activity in epoxidation reactions, and which addresses some or all of the above issues. It is especially preferred to be able to provide such a catalyst which exhibits advantageous performance even when having a reduced loading of titanium therein.

### SUMMARY OF THE INVENTION

According to a first aspect of the present invention there is provided a process for the preparation of a titanium-based catalyst active in epoxidation reactions, which process comprises the steps of:
(a) impregnating a silica carrier with a liquid solution of a titanium compound in an inorganic solvent system, to form an impregnated silica carrier bearing the solution of the titanium compound;
(b) drying the impregnated silica carrier obtained in step (a);
(c) calcining the product obtained in step (b) at a temperature of at most 750° C.; and
(d) silylating the product obtained in step (c), to give a titanium-based catalyst active in epoxidation reactions.

### DETAILED DESCRIPTION OF THE INVENTION

This process provides a simple way of emplacing titanium on a silica carrier by means of an inorganic solvent-based (e.g. water-based) impregnation of soluble titanium compounds, which results in a quick and effective way of preparing a titanium-based catalyst active in epoxidation (hereinafter also referred to as a titanium-based catalyst, a titanium catalyst or an epoxidation catalyst) and wherein the resulting catalyst preserves favourable titanium geometries, that is to say, a significant amount of tetrahedral Ti species.

The process according to the first aspect of the invention may be particularly suitable for quickly producing a batch of epoxidation catalyst in an emergency, e.g. if a problem arises in the production of epoxidation catalyst via a more complicated gas-based process, since the new process can be used in a standard manufacturing system without requiring special dedicated assets.

The process of the invention can be more environmentally friendly than the known methods; the inorganic solvent system ensures that no organic solvents need to be burned off, which would result in the release of CO₂. Furthermore, burning organic solvents can cause temperature runaways which could damage the catalyst or even cause explosions or other safety issues.

Step (a) involves impregnating a silica carrier with a liquid solution of a titanium compound in an inorganic solvent system to form an impregnated silica carrier bearing the solution of the titanium compound.

The inorganic solvent system is inorganic in the sense that it comprises or consists of one or more inorganic solvents. Inorganic solvents are solvents which do not contain carbon.

In an embodiment, the inorganic solvent system comprises water, sulphuric acid, ammonia, or any combination thereof.

In an embodiment, the inorganic solvent system comprises water, which means the liquid solution is an aqueous solution. In an embodiment, the inorganic solvent system comprises water optionally combined with sulphuric acid or ammonia. In an embodiment, water, or an aqueous solution comprising sulphuric acid or ammonia, make up at least 90 wt %, suitably at least 99 wt %, of the inorganic solvent system. In an embodiment, the inorganic solvent system consists of water optionally combined with sulphuric acid or ammonia. In an embodiment, the inorganic solvent system comprises or consists of water and sulphuric acid.

In an embodiment the inorganic solvent system comprises at least 50 wt % water, in particular at least 70 wt % water.

The titanium compound may in principle be any titanium compound which is soluble in the relevant inorganic solvent system. Conveniently, it may for example be a water-soluble titanium compound.

In an embodiment, the titanium compound is a titanium(IV) compound.

In an embodiment, the titanium compound is a titanium complex comprising one or more organic ligands. Examples of such ligands are, for example, a lactate or an oxalate ligand. In an embodiment, the titanium complex is in the form of a salt that can dissociate in water. Advantageously, the titanium complex may be in the form of an ammonium salt. In an embodiment, the titanium compound is titanium(IV) bis(ammonium lactato)-dihydroxide.

In a preferred embodiment, the titanium compound is titanium(IV) oxysulphate.

The solution may be acidic, basic or neutral.

Particularly since the titanium compound can take a range of forms, a range of pH values may be used.

In an embodiment, the solution is acidic. Acidic solutions can have a number of advantages, such as improved solubility of the titanium compound, particularly when the titanium compound is titanium(IV) oxysulphate. Furthermore, in order to improve titanium deposition on silica, silica can be treated with acidic solutions, which can scavenge impurities of the silica carrier, thus minimizing side reactions during catalyst preparation or a subsequent epoxidation reaction. An impregnation using an acidic solution (for example an acidic aqueous solution) of a titanium compound can have a similar effect, without the need for a preceding acidic treatment step, which can limit the required manufacturing steps while preserving the qualities of the resulting catalyst. In an embodiment, the process therefore excludes such a separate pre-treatment step.

In an embodiment, the solution of the titanium compound has a pH of less than 5, less than 4, less than 3, or less than 2. In an embodiment, the solution of the titanium compound has a pH of from 1 to 5, from 1 to 4, from 1 to 3, or from 1 to 2, in particular when the titanium compound is titanium(IV) oxysulphate.

In an embodiment, the solution of the titanium compound has a pH of from 6 to 9, for example from 6 to 8, from 7 to 9, or from 7 to 8, in particular when the titanium compound is a titanium complex comprising one or more organic ligands, such as for example titanium(IV) bis(ammonium lactato)-dihydroxide.

The amount of titanium required in the solution of the titanium compound can be calculated by the skilled person depending on the desired weight percentage of titanium in the final catalyst using routine lab techniques (see Examples 1 and 2).

In an embodiment, the weight percentage of titanium in the solution of the titanium compound is from 0.1 to 10 wt %, for example from 1 to 5 wt %.

The silica carrier may substantially consist of (optionally hydrous) silicon dioxide. However, limited amounts of further compounds, e.g. contaminants, can be present as well, and these can influence the performance of the final catalyst. In an embodiment, the silica carrier used in the present invention comprises at most 1200 ppm of sodium, or at most 1000 ppm of sodium. In an embodiment, the silica carrier comprises at most 500 ppm of aluminium, at most 500 ppm of calcium, at most 200 ppm of potassium, at most 100 ppm of magnesium and/or at most 100 ppm of iron. The amounts are based on amount of carrier.

In an embodiment, the silica carrier is a silica gel. The silica gel carrier can be any carrier derived from a silicon containing gel. In general, silica gels are a solid, amorphous form of hydrous silicon dioxide, distinguished from other hydrous silicon dioxides by their microporosity and hydroxylated surface. Silica gels usually contain three-dimensional networks of aggregated silica particles of colloidal dimensions. They are typically prepared by acidifying an aqueous sodium silicate solution by combining it with a strong mineral acid. The acidification causes the formation of monosilicilic acid (Si(OH)₄), which polymerizes into particles with internal siloxane linkages and external silanol groups. The polymer particles aggregate, thereby forming chains and ultimately gel networks. Silicate concentration, temperature, pH and the addition of coagulants affect gelling time and final gel characteristics such as density, strength, hardness, surface area (SA) and pore volume (PV). The resulting hydrogel is typically washed free of electrolytes, dried and activated. Examples of suitable silica gel carriers are those silica supports available under the trade designations “V432” and “DAVICAT P-732”, from Grace Davison.

In an embodiment, the silica carrier may be a shaped extrudate of silica powder, for example as described in WO2001097967. Shaped extrudates of silica powder differ from silica gel carriers in their manufacturing method and in their physical properties. The high mechanical energy required to form the extrudate imparts high crushing strength and density to the extrudate but can decrease pore volume. A disadvantage of extrudates is that multiple steps are required for obtaining extrudates of suitable strength.

In an embodiment, the silica carrier has a weight average particle size of at most 2.5 mm, at most 2.3 mm, at most 2.0 mm, at most 1.8 mm, at most 1.6 mm, or at most 1.4 mm. In an embodiment, the weight average particle size is at least 0.2 mm, at least 0.4 mm, or at least 0.6 mm. In an embodiment, the weight average particle size is from 0.2 to 2.5 mm, from 0.4 to 2.5 mm, from 0.6 to 2.5 mm, from 0.2 to 2.3 mm, from 0.4 to 2.3 mm, from 0.6 to 2.3 mm, from 0.2 to 2.0 mm, from 0.4 to 2.0 mm, from 0.6 to 2.0 mm, from 0.2 to 1.8 mm, from 0.4 to 1.8 mm, from 0.6 to 1.8 mm, from 0.2 to 1.6 mm, from 0.4 to 1.6 mm, from 0.6 to 1.6 mm, from 0.2 to 1.4 mm, from 0.4 to 1.4 mm, or from 0.6 to 1.4 mm. In an embodiment, the weight average particle size is about 1.3 mm.

In an embodiment, the silica carrier has a surface area (SA) of at least 190 m²/g, at least 200 m²/g, at least 250 m²/g, or at least 300 m²/g. In an embodiment, the silica carrier has a surface area of at most 1000 m²/g, at most 800 m²/g, or at most 500 m²/g. In an embodiment, the surface area is from 190 m²/g to 1000 m²/g, from 200 m²/g to 1000 m²/g, from 250 m²/g to 1000 m²/g, from 300 m²/g to 1000 m²/g, from 190 m²/g to 800 m²/g, from 200 m²/g to 800 m²/g, from 250 m²/g to 800 m²/g, from 300 m²/g to 800 m²/g, from 190 m²/g to 500 m²/g, from 200 m²/g to 500 m²/g, from 250 m²/g to 500 m²/g, or from 300 m²/g to 500 m²/g. In an embodiment, the surface area is about 330 m²/g.

In an embodiment, the silica carrier has a pore volume (PV) of from 0.8 cm³/g to 1.3 cm³/g. In an embodiment, the silica carrier has a pore volume (PV) of from 0.9 cm³/g to 1.3 cm³/g, from 1.0 cm³/g to 1.3 cm³/g, from 1.1 cm³/g to 1.3 cm³/g, from 0.8 cm³/g to 1.2 cm³/g, from 0.9 cm³/g to 1.2 cm³/g, from 1.0 cm³/g to 1.2 cm³/g, or from 1.1 cm³/g to 1.2 cm³/g. In an embodiment, the pore volume is about 1.15 cm³/g.

In an embodiment, step (a) is carried out by using pore volume impregnation type methods. These impregnation methods involve adding solution to a dry carrier in an amount based on the pore volume of the carrier. Examples of these types of impregnation methods include pore volume impregnation, dry impregnation, incipient wetness impregnation and capillary impregnation. The techniques are well-known, and are based on dissolving the active ingredient or its precursor in a solution and adding this solution to a carrier where the capillary action draws the solution into the pores of the carrier. The amount of the added solution is lower or at most equal to the pore volume of the carrier. Further information can be found in “MANUAL OF METHODS AND PROCEDURES FOR CATALYST CHARACTERIZATION” by J. HABER, J. H. BLOCK and B. DELMON, Pure & Appl. Chem., Vol. 67, Nos 8/9, pp. 1257-1306, 1995 (IUPAC).

The use of pore volume impregnation helps to ensure efficient use of titanium, since only the amount of titanium which is desired to be present on the final catalyst needs to be dissolved in the appropriate amount of solvent, after which the entire obtained solution is impregnated onto the silica carrier. This is exemplified in Examples 1 and 2.

In an embodiment, step (a) is carried out by using dynamic impregnation type methods, such as for example circulating solution impregnation. This method involves circulating a solution through a bed of carrier.

In an embodiment, the process according to the first aspect of the invention comprises a further step before step (a), wherein the silica carrier is dried. In an embodiment, the drying method comprises subjecting the silica carrier to a temperature of from 100 to 400° C., such as for example from 200 to 400° C. In an embodiment, the drying is carried out during from 1 to 8 hours. In an embodiment, the drying is carried out in the presence of an inert gas such as nitrogen.

Step (b) involves drying the impregnated silica carrier obtained in step (a). In an embodiment, step (b) comprises subjecting the impregnated silica carrier obtained in step (a) to a temperature of from 100 to 400° C., such as for example from 200 to 400° C., from 100 to 300° C., from 200 to 300° C., from 100 to 300° C., or from 100 to 200° C.

Step (c) involves calcining the product obtained in step (b). The calcination step can fix the titanium on the surface of the silica carrier and can destroy any non-titanium components of the titanium compound present on the carrier.

In an embodiment, calcination of the impregnated carrier in step (c) is carried out by subjecting the product obtained in step (b) to a temperature of at least 500° C., at least 550° C., at least 600° C., at least 650° C., or at least 700° C.

In preferred embodiments, the calcination is carried out at a temperature of at most 600° C., at most 650° C. or at most 700° C.

In an embodiment, the calcination is carried out at a temperature of from 500° C. to 750° C., from 550° C. to 750° C., from 600° C. to 750° C., from 650° C. to 750° C., from 700° C. to 750° C., from 500° C. to 700° C., from 550° C. to 700° C., from 600° C. to 700° C., from 650° C. to 700° C., from 500° C. to 650° C., from 550° C. to 650° C., from 600° C. to 650° C., or from 500° C. to 600° C.

In an embodiment, the calcination is carried out at a temperature of about 550° C., particularly when the titanium compound is a titanium complex comprising one or more organic ligands, such as titanium(IV) bis(ammonium lactato)-dihydroxide.

In an embodiment, the calcination is carried out at a temperature of about 750° C., particularly when the titanium compound is titanium(IV) oxysulphate.

Step (d) involves silylating the product obtained in step (c), to give a titanium-based catalyst active in epoxidation reactions. This may be done by contacting the product obtained in step (c) with a silylating agent.

In an embodiment, the contacting of the product obtained in step (c) with a silylating agent may be done at an elevated temperature, for example of at least 100° C. or at least 150° C., for example at a temperature from 100 to 500° C., from 100 to 450° C., from 100 to 400° C., from 100 to 350° C., from 100 to 300° C., from 100 to 250° C., from 100 to 200° C., from 150 to 500° C., from 150 to 450° C., from 150 to 400° C., from 150 to 350° C., from 150 to 300° C., from 150 to 250° C., or from 150 to 200° C. In an embodiment, it is about 185° C.

Examples of silylating agents which can be used in step (d) include organosilanes, such as e.g. tetra-substituted silanes with C₁-C₃ hydrocarbyl substituents. In an embodiment, the silylating agent is hexamethyldisilazane (HMDS). Examples of specific suitable silylating methods and silylating agents are, for instance, described in U.S. Pat. No. 3,829,392 and U.S. Pat. No. 3,923,843.

According to a second aspect of the present invention there is provided a titanium-based catalyst active in epoxidation, which is obtainable by the process according to the first aspect of the invention.

The catalyst produced by the process according to the first aspect of the invention is different in structure to known titanium-based catalysts, as evidenced by observed differences in properties (see Tables 2 and 3 in the Examples section).

In an embodiment, the weight percentage of titanium present in the catalyst (based on total weight of the catalyst) is in the range of from 0.1 to 10 wt %, for example from 0.1 to 5 wt %, from 0.1 to 4 wt %, from 0.1 to 3 wt %, from 1 wt % to 10 wt %, from 1 wt % to 5 wt %, from 1 wt % to 4 wt %, from 1 wt % to 3 wt %, from 1.5 wt % to 10 wt %, from 1.5 wt % to 5 wt %, from 1.5 wt % to 4 wt %, from 1.5 wt % to 3 wt %, from 2 wt % to 10 wt %, from 2 wt % to 5 wt %, from 2 wt % to 4 wt %, from 2 wt % to 3 wt %, from 3 wt % to 10 wt %, from 3 wt % to 5 wt %, or from 3 wt % to 4 wt %.

In an embodiment, titanium is the only metal present in the catalyst.

In an embodiment, the weight percentage of titanium in the catalyst is about 4 wt %, particularly when the catalyst has been obtained by using titanium(IV) bis(ammonium lactato)-dihydroxide as the titanium compound in the method according to the first aspect of the invention.

In an embodiment, the weight percentage of titanium in the catalyst is from 2 to 3 wt %, particularly when the catalyst has been obtained by using titanium(IV) oxysulphate as the titanium compound in the method according to the first aspect of the invention.

In a preferred embodiment of the present invention, when the catalyst has been obtained by using titanium(IV) oxysulphate as the titanium compound in the method according to the first aspect of the invention, then the weight percentage of titanium in the final catalyst is about 2 wt %.

According to a third aspect of the present invention there is provided a process for the preparation of an epoxide, which process comprises contacting a hydroperoxide and an alkene with a titanium-based catalyst prepared in accordance with the process according to the first aspect of the invention, and withdrawing a product stream comprising an epoxide and an alcohol and/or water.

As mentioned above, it is well known in the art to produce epoxides by epoxidation of the corresponding alkene using a hydroperoxide as the source of oxygen.

In an embodiment, the alkene is propene and the formed epoxide is propylene oxide.

The hydroperoxide can for example be hydrogen peroxide or an organic hydroperoxide. In an embodiment, the hydroperoxide is ethylbenzene hydroperoxide, tert-butyl hydroperoxide or cumene hydroperoxide.

In an embodiment, the hydroperoxide is ethylbenzene hydroperoxide and the formed alcohol is 1-phenyl ethanol.

In an embodiment, the process further comprises dehydration of 1-phenyl ethanol to obtain styrene.

In an embodiment, the hydroperoxide is tert-butyl hydroperoxide, forming tert-butanol. In an embodiment, tert-butyl hydroperoxide is reacted with propene in the process according to the third aspect of the invention, forming tert-butanol and propylene oxide. In an embodiment, tert-butanol is subsequently etherified into methyl tert-butyl ether (MTBE).

In an embodiment, the hydroperoxide is cumene hydroperoxide, which can optionally be formed by reacting cumene with oxygen or air. In an embodiment, cumene hydroperoxide is reacted with propene in the process according to the third aspect of the invention, forming 2-phenyl propanol and propylene oxide.

The conditions for the process for the preparation of an epoxide according to the third aspect of the present invention are those conventionally applied. For propene epoxidation reactions with the help of ethylbenzene hydroperoxide, typical reaction conditions include temperatures of 50 to 140° C., for example 70 to 120° C., and pressures up to 80 bar, with the reaction medium being in the liquid phase.

The invention is further illustrated by the following Examples.

### EXAMPLES

### Experimental Methods

### Pore Volume & Surface Area

Pore volume is measured together with surface area, using the same method, namely ASTM D4567-03(2008): Standard Test Method for Single-Point Determination of Specific Surface Area of Catalysts and Catalyst Carriers Using Nitrogen Adsorption by Continuous Flow Method.

### Particle Size

The particle size distributions were determined using the Camsizer (Retsche technology Laser Optik Systeme Germany). Accordingly, all particle sizes herein are volume based particle sizes obtained by dynamic image analysis.

### Example 1

### Synthesis of Catalyst A

A reference silica carrier (Grace P543 silica spheres, with nominal properties as set out in Table 1 below) was loaded with 4 wt % Ti using titanium(IV) bis(ammonium lactato)-dihydroxide as a titanium source.

The impregnation solution was obtained by starting from a commercially available starting solution of titanium(IV) bis(ammonium lactato)-dihydroxide in water. This starting solution was analysed by ICP (Inductively Coupled Plasma spectroscopy) and found to contain 7.6 wt % titanium.

The starting solution was diluted with an appropriate amount of water to obtain an impregnation solution. For impregnation of 150 grams of carrier, the impregnation solution was obtained by starting from 78.98 grams of the titanium(IV) bis(ammonium lactato)-dihydroxide starting solution, which contained 6 g of titanium, and adding water up to a total volume of 180 ml.

The entire obtained clear solution was then impregnated on 150 grams of freshly dried carrier by means of pore volume impregnation.

The carrier was rolled for about 1 hour and the next step was to dry the impregnated carrier with a dryer for 1 hour at 120° C. under atmospheric pressure.

Calcination of the resulting material was conducted at 550° C. for 105 minutes.

Gas phase silylation of the obtained product was conducted in an automated bench-scale unit. Preheated hexamethyldisilazane (HMDS) gas with a purge stream of 1.4 Nl/hr nitrogen gas was led over 75 gram of the product obtained from the calcination step at 185° C. The boiling point of HMDS is 126° C. HMDS was dosed at a rate of 18 g/hr. The exothermic silylation reaction was monitored by four thermocouples placed at different heights in the catalyst bed. An exotherm of 25° C. was observed subsequently at all thermocouples within 30 minutes. After 2 hours the HMDS dosage was stopped. Excess HMDS was stripped with 3 Nl/hr at 185° C. for an hour. Excess HMDS and by-product ammonia were vented with the nitrogen stream and the ammonia was neutralized in a NaOH scrubber. After cooling down to room temperature, 78.2 gram of Catalyst A was obtained.

TABLE 1
Nominal Properties of the Reference Silica
Carrier, Grace P543 Silica

| | |
|---|---|
| Surface Area (SA) | 330 m²/g |
| Pore Volume (PV) | 1.15 cm³/g |
| Particle size | Distribution: 0.6-2.0 mm |
| | Average particle size: 1.3 mm |
| Porosity as measured by | 68.6% |
| Hg intrusion | |
| Impurities | none |

### Example 2

### Synthesis of Catalyst B

The same silica carrier as used in Example 1 was loaded with 2 wt % Ti using titanium oxysulphate as a titanium source.

The impregnation solution was obtained by starting from a commercially available starting solution of titanium oxysulphate in dilute sulphuric acid. This starting solution was analysed by ICP and found to contain 4.6 wt % titanium.

The starting solution was diluted with the appropriate amount of water to obtain an impregnation solution. For impregnation of 150 grams of carrier, the impregnation solution was obtained by starting from 22.78 grams of titanium oxysulphate starting solution, which contained 3 g of titanium, and adding water up to a total volume of 180 ml.

The entire obtained clear solution was then impregnated on 150 grams of freshly dried carrier by means of pore volume impregnation.

The carrier was rolled for about 1 hour and the next step was to dry the impregnated carrier with a dryer for 1 hour at 120° C. under atmospheric pressure.

Calcination of the resulted material was conducted at 750° C. for 2 hours.

Gas phase silylation was conducted on 75 gram of the obtained product in the same manner as described for Example 1, resulting in 78.2 gram of Catalyst B.

### Example 3 (Comparative)

### Synthesis of Comparative Catalyst C

Comparative Catalyst C was prepared by loading the same silica carrier as used in Examples 1 and 2 with 4 wt % Ti, in accordance with the known general teaching of EP 0345856 A using gas phase impregnation of titanium tetrachloride onto a silica carrier, followed by calcination at 600° C. and hydrolysis.

This was followed by gas phase silylation of 75 gram of the obtained product in the same manner as described for Examples 1 and 2.

### Example 4

### Comparison of Catalysts A, B and C in the Epoxidation of 1-Octene

In the 1-octene batch test, 50 ml of a mixture containing 7.5 wt % ethylbenzenehydroperoxide (EBHP) and 36 wt % 1-octene in ethylbenzene (EB) was allowed to react with 1 g of epoxidation catalyst at 40° C. while being mixed thoroughly.

After 1 hour, the flask with the reaction mixture was cooled in ice/water to end the reaction and the reaction product was analysed by titration, spectroscopically or by GC. Titration was carried out shortly after ending the test, as the reaction will still proceed at slower pace.

As can be seen from Table 2 below, Catalysts A and B, which were produced in accordance with the invention, have an acceptable level of selectivity to comparative Catalyst C, which was produced using gas phase impregnation.

TABLE 2
Performance Data of Different Ti-based Catalysts
Active in Epoxidation Reactions

| | | Activity per | OO/EBHP |
|---|---|---|---|
| | Ti content | Ti site (mole | selectivity |
| | (wt %) | EBHP/mole Ti) | (%) |
| Catalyst A | 4 | 1.07 | 83.44 |
| Catalyst B | 2 | 2.62 | 86.40 |
| Comparative | 4 | 3.51 | 93.8 |
| Catalyst C | | | |

Catalyst B (containing 2 wt % Ti and prepared using titanium oxysulphate) is particularly beneficial, because it allows emplacement of up to 50% lower Ti loadings (e.g. 2 wt % rather than 4 wt %) while maintaining an acceptable selectivity, which can further lower the release of noxious gases such as SO₂, and can lower the required volumes of acidic solutions of Ti salts. Compared to comparative Catalyst C, which was produced using gas phase impregnation of titanium tetrachloride, it also eliminates formation of corrosive HCl gases, which are very corrosive especially when combined with high temperature.

### Example 5

### Comparison of Catalysts A, B and C in the Epoxidation of Propylene

Epoxidation reaction of propene was carried out with EBHP/EB feed and propylene at a pressure of 40 bar. The experiment is carried out at a WHSV of 16.0 g/gh. About 0.4 g of crushed and sieved catalyst was loaded in the reactor tube. Reaction temperature was kept constant for 222 h at 70 deg C.

As can be seen from Table 3 below, Catalysts A and B, which were produced in accordance with the invention, also have an acceptable level of selectivity to comparative Catalyst C, which was produced using gas phase impregnation.

TABLE 3
Performance Data of Different Ti-based Catalysts
Active in Epoxidation Reactions

| | | Activity per | PO/EBHP |
|---|---|---|---|
| | Ti content | Ti site (mole | selectivity |
| | (wt %) | EBHP/mole Ti) | (%) |
| Catalyst A | 4 | 1.84 | — |
| Unsilylated | | | |
| Catalyst B | 2 | 3.84 | 98.0 |
| Comparative | 4 | 6.24 | 99.0 |
| Catalyst C | | | |

As also seen in Example 4, in Example 5, Catalyst B (containing 2 wt % Ti and prepared using titanium oxysulphate) is again particularly beneficial, because it allows emplacement of up to 50% lower Ti loadings (e.g. 2 wt % rather than 4 wt %) while maintaining an acceptable selectivity, which can further lower the release of noxious gases such as SO₂, and can lower the required volumes of acidic solutions of Ti salts. Compared to Comparative Catalyst C, which was produced using gas phase impregnation of titanium tetrachloride, it also eliminates formation of corrosive HCl gases, which are very corrosive especially when combined with high temperature.

### Example 6

### Comparison of Structure of Catalyst Precursors

Unsilylated catalyst precursors D and E were prepared according to the general procedures of Example 2 and Comparative Example 3, respectively, except without the final silylation step.

UV-VIS spectra were recorded for each of the unsilylated catalyst precursors. These spectra can provide information about the geometries of the Ti centres present on the catalyst. In particular, Ti centres in tetrahedral geometry will result in a band around 210 nm, while Ti centres in octahedral geometry will result in a band around 235 nm.

The relative intensity of the bands at these wavelengths was measured for each of the unsilylated catalyst precursors, to give an indication of the proportions of the different Ti geometries present in these catalyst precursors.

These data are presented in Table 4 below, in the form of the ratios between the intensities of the bands at 210 and 235 nm.

TABLE 4

| Catalyst precursor | Ratio 210/235 nm |
|---|---|
| D (in line with Ex. 2) | 0.21 |
| E (in line with Comp. Ex. 3) | 0.73 |

As can be seen from these data, catalyst precursor D (prepared according to the general procedures of Example 2 of the invention) has a lower 210 nm:235 nm ratio, i.e. a lower proportion of tetrahedral Ti centres and a higher proportion of octahedral Ti centres, than catalyst precursor E (prepared according to Comparative Example 3).

Catalyst precursor E is made by a chemical vapour deposition method, which by its nature, will deliver a high number of Ti tetrahedral sites.

However, whilst Catalyst precursor D has a lower number of tetrahedral Ti sites than Catalyst precursor E produced by gas phase impregnation of titanium tetrachloride onto a silica carrier, unlike the titania-silica catalysts prepared in U.S. Pat. No. 3,829,329 and EP1005907, it is apparent that the manufacturing procedure of the present invention allows significant preservation of highly active tetrahedral species.

I claim:

1. A process for the preparation of a titanium-based catalyst active in epoxidation reactions, which process comprises the steps of:
(a) impregnating a silica carrier with a liquid solution of a titanium compound in an inorganic solvent system, to form an impregnated silica carrier bearing the solution of the titanium compound;
(b) drying the impregnated silica carrier obtained in step (a);
(c) calcining the product obtained in step (b) at a temperature of at most 750° C.; and
(d) silylating the product obtained in step (c), to give a titanium-based catalyst active in epoxidation reactions.

2. The process of claim 1, wherein the inorganic solvent system comprises water.

3. The process of claim 2, wherein the inorganic solvent system comprises water combined with sulphuric acid or ammonia.

4. The process of claim 1, wherein the titanium compound is a titanium(IV) compound.

5. The process of claim 1, wherein the titanium compound is a titanium complex comprising one or more organic ligands.

6. The process of claim 5, wherein the titanium compound is titanium(IV) bis(ammonium lactato)-dihydroxide.

7. The process of claim 1, wherein the titanium compound is titanium(IV) oxysulphate.

8. The process of claim 1, wherein the solution of the titanium compound has a pH of from 6 to 9.

9. The process of claim 1, wherein the solution of the titanium compound has a pH of less than 5.

10. The process of claim 1, wherein step (a) is carried out by pore volume impregnation.

11. A titanium-based catalyst active in epoxidation, which is obtainable by the process according to claim 1.

12. A process for the preparation of an epoxide, which process comprises contacting a hydroperoxide and an alkene with a titanium-based catalyst prepared in accordance with the process of claim 1, and withdrawing a product stream comprising an epoxide and an alcohol and/or water.

13. The process of claim 12, wherein the alkene is propene and the epoxide is propylene oxide.

14. The process of claim 12, wherein the hydroperoxide is ethylbenzene hydroperoxide and the alcohol is 1-phenyl ethanol.

15. The process of claim 14, wherein the process further comprises dehydration of 1-phenyl ethanol to obtain styrene.
